(19) United States
(12) Patent Application Publication
(10) Pub. No.: US 2011/0015134 A1
(43) Pub. Date: Jan. 20, 2011

(54) Method of treatment for early stage cancer

(76) Inventor: Michael W. Retsky, Trumbull, CT (US)

Correspondence Address: Michael W. Retsky, 237 Strobel Road, Trumbull, CT, 06611, US

(21) Appl. No.: 12/924,162

(22) Filed: Sep. 22, 2010

Related U.S. Application Data
(63) Continuation-in-part of application No. 12/148,089, filed on Apr. 16, 2008.
(60) Provisional application No. 60/923,649, filed on Apr. 16, 2007.

(51) Int. Cl. A61K 38/00; A61K 31/421; A61K 31/4025; A61K 31/192; A61K 31/19; A61K 31/405; A61K 31/407; A61K 31/195; A61K 31/12; A61K 31/5415; A61K 31/542; A61K 31/60; A61K 31/167; A61K 31/138; A61K 31/4523; A61K 31/55; A61K 31/4196; A61K 31/566; A61P 35/00

(52) U.S. Cl. 514/19.3; 514/374; 514/422; 514/570; 514/569; 514/420; 514/411; 514/567; 514/682; 514/226.5; 514/224.5; 514/161; 514/162; 514/164; 514/630; 514/648; 514/324; 514/217.08; 514/383; 514/171

(57) ABSTRACT

This patent describes a method and materials to treat cancer diagnosed at an early stage, particularly breast cancer. It considers that metastatic breast cancer growth includes periods of dormancy, that surgery to remove a primary tumor can induce metastatic growth, and that women with Down Syndrome rarely get breast cancer. It elevates the level of an antiangiogenic drug produced by chromosome 21 preferably Endostatin in plasma preferably at least one day prior to surgery and kept at that high level preferably indefinitely. In one embodiment, the therapy specifically excludes drugs that significantly inhibit the VEGF pathway since that is important for wound healing. This method will prevent results of surgery from stimulating tumor growth and angiogenesis of micrometastatic disease that is much easier to prevent than control after the fact. This can be done indefinitely since there is no acquired resistance that develops, as happens in most cancer therapies.

### CROSS-REFERENCE TO RELATED APPLICATIONS

This application is a continuation-in-part of U.S. application Ser. No. 12/148,089, filed Apr. 16, 2008, which claims the benefit of U.S. Provisional Patent Application No. 60/923,649, filed on Apr. 16, 2007, the contents of each of which is incorporated herein by reference in its entirety.

### BACKGROUND DESCRIPTION

This invention relates to treatment for early stage cancer with particular relevance to breast cancer. While the mortality rate has been dropping in recent years, breast cancer is diagnosed in 120,000 women and still kills over 40,000 yearly in the US. When breast cancer is first diagnosed, the patient is given a work-up to determine if there is any evidence of distant metastases. If there is no overt sign of distant metastases, the stage is considered early. If there is evidence of distant metastases at diagnosis or at any time later in the disease process the stage is called late.

There is determined effort to detect breast cancer at the earliest possible time since outcome after just surgery is more often favorable than it is compared to detection later. For example, women detected with a primary tumor of 1 cm in size and no axillary lymph nodes involved with cancer can expect 90% probability of cure after only removal of the primary tumor. On the other hand, a patient with 5 cm tumor and 10 lymph nodes with cancer can expect only 10% probability of cure with simple surgical removal of the primary tumor. Patients rarely die from the primary tumor. The risk is for later relapse of the cancer in an organ that is not so easy to treat such as lung, liver, brain, or bone. Over 90% of new cases of breast cancer are diagnosed in the early stage.

After surgery to remove the primary tumor, therapy called surgical adjuvant therapy or just adjuvant therapy is often administered to help prevent or delay any possible appearance of distant metastases in the next 15-20 years. It may be in the form of cytotoxic chemotherapy or less toxic hormone therapy. There are well-established means and guidelines to determine which if either or both of these therapies is indicated for any particular patient.

Treatment for early stage breast cancer too often ultimately fails in that metastatic disease is discovered within 15-20 years after initial diagnosis. Adjuvant chemotherapy improves absolute cure rates by up to 15%. Hormone therapy has approximately the same benefit.

Treatment for metastatic disease is mainly palliative in that long term survival with that disease state is very rare. The median time of survival after relapse from early stage breast cancer is two years. There is an urgent need for improved treatments for early stage breast cancer that are far more effective in preventing relapses for long periods of time—hopefully until the person dies of another disease or old age. Based on the experience over the past few decades, we are more likely to make an impact by learning how to more effectively prolong remission in early stage breast cancer than we are in learning how to eradicate a tumor that is macroscopic in size.

Thirty years ago Judah Folkman (1933-2008) founded the field of tumor angiogenesis—which describes a process by which a cancer acquires a blood supply from the host. Without this blood supply, a cancer cannot grow more than a millimeter or so in size. After this blood supply is established, tumor growth leading to a lethal size of approximately 1 liter can then happen.

Studies of breast cancer tumor growth and angiogenesis suggest that when a person is diagnosed with early stage breast cancer, it is rare that any sites of metastatic disease deposits have achieved angiogenesis. That is, there are often many distant dormant single cancer cells and distant dormant small cancer deposits in the person other than the primary tumor that have not progressed beyond a mm or so in size.

A surprising finding is that surgery to remove the primary tumor often kick-starts growth of the dormant cells and avascular micrometastases. Most relapses occur within the first 5 years after surgery. These are mostly events that are triggered into growth from surgery. It has been suggested that one of the side effects of surgical wounding is to stimulate division of dormant single malignant cells and stimulate angiogenesis of dormant micrometastases. The latter is most apparent for the premenopausal node-positive population. According to these reports, 20% of premenopausal node-positive patients undergo surgery-induced angiogenesis and over half of all relapses in breast cancer are accelerated by surgery.

These effects reduce the benefit of early detection. Most persons derive benefit from early detection since they will be diagnosed with less extensive disease but paradoxically other persons will relapse and die earlier as an unfortunate consequence of early detection. This is most apparent in young women.

Not coincidentally, adjuvant chemotherapy works best by far for premenopausal patients who are node-positive. According to some theories, the reason for this is that the sudden metastatic tumor growth just after surgery produces a chemosensitive window just at the time when adjuvant therapy was empirically found to be most effective. One implication is that surgery produces a disruption and acceleration of disease and then adjuvant chemotherapy is used to counteract the effects of the disruption.

In 2005, data was analyzed from an adjuvant hormone therapy trial comparing Tamoxifen and Arimidex. As was reported, hormone therapy mainly acts to suppress relapses that would have occurred in the first 5 years after surgery for hormone receptor positive patients. Tamoxifen, the most frequently used hormone therapy drug, is given only in the first five years after surgery. After that time, Tamoxifen has no demonstrated value. One way of interpreting these data is that adjuvant hormone therapy, like adjuvant chemotherapy, functions to counteract surgery induced growth of micrometastatic disease.

It has been proposed that antiangiogenic drugs given when disease is still microscopic would be very helpful but that this treatment should best be initiated before surgery. After that, “the horse is out of the barn.” It is far more difficult to reverse angiogenesis after it is established than it is to prevent it from happening before it occurs.

That presents a serious problem since it is widely accepted that wound healing after surgery highly depends on angiogenesis to remodel and rebuild tissue. So it would appear that starting an antiangiogenic therapy before surgery to prevent micrometastases from escaping dormancy would interfere with wound healing after primary tumor removal. This seems to preclude using an antiangiogenic therapy before surgery. What is needed is a possible way around this apparent impasse.

Anesthetic and analgesic agents are emerging as potential factors influencing postoperative relapse in patients undergoing surgery to remove primary tumors, presumably by affecting the perioperative angiogenic potential in the patient. Moreover, conditions during surgery which increase the stress response (e.g. blood transfusion or hypothermia) may affect the perioperative angiogenic potential in the patient as well.

It would be very important if a way could be found to treat early stage breast cancer with an effective antiangiogenic drug for an indefinite time starting before surgery to remove the primary but yet not interfere with wound healing resulting from the surgery. The prior art lacks a method of preventing angiogenesis of dormant micrometastases initiated before primary surgery yet without interfering with wound healing and having essentially no toxicity while used over very extended periods of time. Preferably, such a strategy would also take into account the effects of anesthetic and analgesic agents on the perioperative angiogenic potential of the patient. The present invention overcomes the limitations of the prior art by providing, in various embodiments, treatment methods designed to prevent metastatic relapse which minimize the short and long term effects of surgery and can be safely administered long-term.

### SUMMARY OF THE INVENTION

The invention describes methods and materials to treat cancer diagnosed at an early stage. Particular reference is to breast cancer but it may also apply to other cancers such as lung, prostate, melanoma, osteosarcoma, ovarian, and cervical. It might also apply to colon cancer and other gastrointestinal cancers although that is somewhat less likely. To be most effective the antiangiogenic drugs described must be initiated preferably at least one day prior to surgical removal of the primary tumor. This will prevent results of surgery from stimulating angiogenesis of micrometastatic disease that is much easier to prevent than control after the fact.

The antiangiogenic drugs described are mostly chromosome 21 based since Down Syndrome persons have trisomy 21 and very rarely develop breast cancer. They also do not have wound healing difficulty. In one embodiment of the invention, the use of VEGF inhibiting drugs at least before and shortly after surgery is avoided since we know the VEGF angiogenic pathway is key in wound healing. Chromosome 21 proteins are non-toxic when used for very extended periods of time based on the Down Syndrome experience. There are at least 283 genes on chromosome 21 that may produce proteins. All these proteins are potential candidates although Endostatin and NC1 are most likely to be effective. Another drug that would work is Endostar, a variant of Endostatin that is currently used for lung cancer in China. Additional angiogenesis inhibitors useful for practicing the present invention include protein isoforms 2, 3 or 4 encoded by the DSCR1 gene on chromosome 21, and the proteins encoded by the DYRK1A and ADAMTS genes on chromosome 21.

The invention further describes how to continue preventing angiogenesis with a non-toxic therapy. This can be done indefinitely since there is no acquired resistance that develops with an endogenous protein from chromosome 21. Other cancer therapies eventually fail due to acquired drug resistance.

Administration of an antiangiogenic prior to surgery may or may not eradicate the micrometastases even if given over a long time but it could prevent growth indefinitely. The advantages are important. First, adjuvant therapy might prove to be unnecessary. Second, avascular dormancy is naturally very stable and would be far easier to maintain long term with a low toxicity angiogenesis inhibitor in comparison to eradicating metastases with chemotherapy, radiation, surgery or antiangiogenic therapy after they start to grow. Third, wound healing would be unimpaired. Fourth, this therapy could be continued for ensuing years at appropriate levels and may prevent future relapses for all early stage breast cancer patients. Fifth, this therapy takes full advantage of early detection and there will be no paradoxical disadvantage to anyone diagnosed early. Sixth, mortality from breast cancer will be reduced. Seventh, this therapy could be easily implemented in developing countries where access to medical specialists, imaging equipment, well-equipped pathology labs and costly drugs is limited.

In one embodiment, the present invention provides a method for preventing relapse in a cancer patient undergoing surgery comprising administering a first drug that has antiangiogenic properties starting before the initial surgical incision and continuing at least until the end of surgery; and administering a second drug that has antiangiogenic properties starting within two months past the end of surgery and continuing for at least one year.

In a related embodiment, the first drug that has antiangiogenic properties is a non-steroidal anti-inflammatory drug (NSAID). The NSAID may be, without limitation, acetaminophen, aspirin, a propionic acid derivative such as ibuprofen, naproxen, fenoprofen, ketoprofen, flurbiprofen or oxaprozin, an acetic acid derivative such as indomethacin, sulindac, etodolac, ketorolac, diclofenac or nabumetone, an enolic acid derivative such as piroxicam, meloxicam, tenoxicam, droxicam, lornoxicam, or isoxicam, a fenamic acid derivative such as mefenamic acid, meclofenamic acid, flufenamic acid, tolfenamic acid, or a selective COX-2 inhibitor such as celecoxib, rofecoxib, valdecoxib, parecoxib, lumiracoxib or etoricoxib. Preferably the NSAID functions as a non-selective cyclooxygenase (COX) inhibitor, thereby inhibiting the synthesis of prostaglandins. When the first drug is an NSAID, ketorolac (a racemic mixture of (S)-(−) and (R)-(+) isomers) is preferred. Ketorolac may be administered by any route; however, intravenous administration at doses of 20 mg or 30 mg are preferred.

In a related embodiment, first drug that has antiangiogenic properties is selected from endostatin, angiostatin, Endostar, tumstatin and thrombospondin. Preferably the first drug is endostatin or Endostar and is taken to a level in plasma greater than 24.36 ng/ml before the start of surgery.

In another embodiment, the second drug that has antiangiogenic properties is a non-steroidal anti-inflammatory drug (NSAID). The NSAID may be, without limitation, acetaminophen, aspirin, a propionic acid derivative such as ibuprofen, naproxen, fenoprofen, ketoprofen, flurbiprofen or oxaprozin, an acetic acid derivative such as indomethacin, sulindac, etodolac, ketorolac, diclofenac or nabumetone, an enolic acid derivative such as piroxicam, meloxicam, tenoxicam, droxicam, lornoxicam, or isoxicam, a fenamic acid derivative such as mefenamic acid, meclofenamic acid, flufenamic acid, tolfenamic acid, or a selective COX-2 inhibitor such as celecoxib, rofecoxib, valdecoxib, parecoxib, lumiracoxib or etoricoxib. When the second drug is an NSAID, Aspirin is preferred. Preferably, the NSAID is administered for a period beginning within 1, 2, 3, 4, 5, 6 or 7 weeks past the end of surgery and continuing for at least 1, 2, 3, 4, or 5 years. The NSAID may be administered according to any known administration regimen and may be administered continuously or periodically. Preferably, the NSAID is administered at a daily dosage.

In yet another embodiment, the second drug that has antiangiogenic properties is a selective estrogen receptor modulator (SERM) (e.g. tamoxifen, raloxifene, toremifene) or an aromatase inhibitor (e.g. anastrozole). Preferably the SERM or aromatase inhibitor is administered in an adjuvant capacity (i.e. after the end of surgery) and preferably continues for at least 5 years post-surgery. Administration of the SERM or aromatase inhibitor may be according to any current hormone adjuvant protocol known in the art.

Any SERM known in the art may be used in the invention, including, without limitation, clomiphene, femarelle, ormeloxifene, raloxifene, tamoxifen, toremifene, afimoxifene, arzoxifene, bazedoxifene, and lasofoxifene. Preferably, the SERM is selected from tamoxifen, raloxifene and toremifene. Most preferably the SERM is tamoxifen. Any aromatase inhibitor known in the art may be used in the invention, including, without limitation, anastrozole, exemestane, letrozole, vorozole, formestane, and fadrozole. Preferably, the aromatase inhibitor is anastrozole, exemestane or letrozole, most preferably anastrozole. Preferably, the patient undergoing hormone adjuvant treatment is a post-menopausal female with a hormone receptor (ER or PR)-positive tumor.

In a related embodiment, the first drug that has antiangiogenic properties and the second drug that has antiangiogenic properties are each NSAIDs. In a preferred embodiment, the first drug is ketorolac and second drug is selected from Ibuprofen, Naproxen, Fenoprofen, Ketoprofen, Flurbiprofen, Oxaprozin, Indomethacin, Sulindac, Etodolac, Ketorolac, Diclofenac, Nabumetone, Piroxicam, Meloxicam, Tenoxicam, Droxicam, Lornoxicam, Isoxicam, Mefenamic acid, Meclofenamic acid, Flufenamic acid, Tolfenamic acid, Aspirin, and Acetaminophen. In another preferred embodiment, the first drug is ketorolac and the second drug is ketorolac.

In another embodiment, the first and second drugs are each SERMs, are each aromatase inhibitors or are each endostatin, angiostatin, Endostar, tumstatin or thrombospondin.

In a related embodiment, if the second drug is a SERM, aromatase inhibitor, or NSAID, a third drug having antiangiogenic properties is administered to the patient for a period starting within two months past the end of surgery and continuing for at least one year. If the second drug is a SERM, the third drug is selected from an aromatase inhibitor and an NSAID. If the second drug is an aromatase inhibitor, the third drug is selected from a SERM and an NSAID. If the second drug is an SAID, the third drug is selected from a SERM and an aromatase inhibitor.

In a related embodiment, opioids, in particular morphine, are not administered to the cancer patient undergoing the treatment methods of the present invention during the perioperative period and preferably for at least one week thereafter. The term “perioperative” is defined in the PDR Medical Dictionary, Second Edition, (Medical Economics Company, 2000), as “around the time of operation.” The perioperative period is characterized by a sequence including the time preceding an operation when a patient is being prepared for surgery (“the preoperative period”), followed by the time spent in surgery (“the intraoperative period”), and by the time following an operation when the patient is closely monitored for complications while recovering from the effects of anesthesia (“the postoperative period”).

In another embodiment, no drugs with VEGF inhibiting activity are co-administered to the patient during the perioperative period.

In a related embodiment, cancer patients undergoing the treatment methods of the present invention do not undergo blood transfusion during the perioperative period.

In a related embodiment, cancer patients undergoing the treatment methods of the present invention are not hypothermic during the perioperative period.

In another embodiment, any of the treatment methods described above may comprise administration of the active agent(s) according to a metronomic schedule. By metronomic schedule, it is meant that the active agent is administered at a dose lower than the maximum tolerated dose which is nevertheless effective to prevent or delay the growth of cancer, preferably at frequent intervals and without extended active-agent-free periods. Over a given treatment cycle, the metronomic schedule may be such that 80% less (and preferably 70%, 60%, 50%, 40%, 30%, 20% or less) of the drug is administered than would have been at a regimen which employs the maximum tolerated dose. The drug may be administered in a range from continuous infusion, to daily, weekly or anywhere therebetween. Such a schedule is to be contrasted with the maximum tolerated dose regime normally used for cancer therapeutic agents, where administration may consist of high dose(s) administered over short periods followed by active-agent free periods (often 2-3 weeks) where no active agent is administered in order to allow recovery of non-cancer cells which are killed by the high doses.

### BRIEF DESCRIPTION OF THE DRAWINGS

FIG. 1 is a schematic outline of how early stage breast cancer is currently treated indicating prior art.

FIG. 2 is a schematic outline of how early stage breast cancer is treated according to the invention.

### DETAILED DESCRIPTION OF THE PRESENTLY PREFERRED EMBODIMENTS

In order to explain clinical breast cancer data, sometimes surgery to remove a primary breast tumor induces division in distant dormant single metastatic cells and also induces angiogenesis of distant dormant micrometastases. Over half of all relapses are accelerated by these processes.

The undisturbed half-life of avascular micrometastases in breast cancer is 2 years and the undisturbed half-life of single dormant cells is 1 year. This suggests that the avascular dormant state is the more stable of the two dormant states. Efforts to prolong the natural tendency of dormancy of disease in these early states, especially the pre-angiogenic state, could be pursued as one method to reduce cancer mortality.

It has been reported that at the time of detection of early stage breast cancer, the state of any metastatic disease is rarely past the point of angiogenesis. Mostly the sites of potential future disease are dormant as single non-dividing cells or as avascular micrometastases. There is balanced cell division and cell death within an avascular micrometastasis so the net volume does not change appreciably. As indicated, surgery to remove the primary tumor sometimes initiates cell growth and angiogenesis. Knowing this, it seems logical that the best time to use an antiangiogenic drug is to start before surgery. If started after that, the use of an antiangiogenic drug will be more or less moot since the damage has already been done. Once the tumor has a blood supply, it has proven difficult to prevent it from growing to a size that can cause life-threatening problems. If we want to consider an effective antiangiogenesis therapy, the best time to start is before surgery.

However, that will cause a problem since it is well known that angiogenesis is necessary in the wound healing process after surgery. It would be very useful if a method to prevent surgery-induced angiogenesis of distant dormant micrometastases could be found that does not interfere with the wound repair process.

There is additional relevant information. Endostatin is the C-terminus fragment of collagen XVIII (blood clotting function) and is a very robust inhibitor of angiogenesis. The mechanism is thought to be an inhibition of endothelial cell migration and also to induce apoptosis—or programmed cell death. It is endogenous to all humans and is thus quite non-toxic. In fact, it has never been shown to exhibit toxicity at any level at any concentration. This is unique in the history of the FDA testing program. Naturally, it has been suggested that Endostatin should be given to all healthy persons. That would effectively eliminate cancer.

In support of that argument, it has been pointed out that persons with Down Syndrome rarely have breast cancer (10-25 fold less than age-matched normals according to Benard et al) and that they also have an elevated level of Endostatin. This is correlated to the genetic defect in that Down Syndrome (“DS”) persons have approximately an extra copy of chromosome 21. Normal persons have two copies of chromosome 21. DS have between two and three copies of chromosome 21. This is referred to as trisomy 21.

Accordingly, there are at least 283 protein encoding genes in this chromosome which corresponds to approximately 1% of the human DNA. This chromosome that causes the retardation also codes for collagen XVIII so, on average, Down Syndrome persons have more Endostatin than normals. The ratio is approximately 1.8 according to Zorick et al and 1.48 according to Greene et al.

DS often have congenital heart disease that is repairable with surgery so there are data on wound healing. The results of surgery to repair complete atrioventricular septal defect in 476 patients, 71.6% of who were DS, and the remainder normal have been reported. There was 30 day mortality of 4.9% in the DS and 5.6% in the normals. There was pulmonary hypertension more often among DS than normals but there was no difference in operational strategy or timing of repair. It was concluded that the presence of DS was not a risk factor for surgical repair of complete atrioventricular septal defect.

Endostatin has proven very difficult to manufacture in any significant quantities. It has not made an impact in reducing cancer as was originally widely hoped. This has been attributed to the high difficulty and cost of manufacturing and resulting very small availability of the drug. In limited tests, it has occasionally and dramatically stabilized disease in a few otherwise hopeless cases but as mentioned has not made the hoped-for impact in humans.

A molecule very similar to Endostatin called Endostar has been manufactured in significant quantities by a company in China. This drug has been tested and found to be twice as effective as any Endostatin ever tested. Endostar is currently used in China for late stage lung cancer patients but is not currently approved for use in the US.

Endostatin can maintain tumors in a state of dormancy although the half-life is short so Endostatin is best utilized with prolonged delivery using mini-osmotic pumps or slow release encapsulation systems. Reportedly, results are best when the drug is administered as early as possible. No evidence of drug resistance has been seen.

It has been suggested that 1.6 or 1.7 fold increase of Endostatin relative to average normal level will prevent angiogenesis. Others have suggested that only 30% more Endostatin than normal will effectively prevent angiogenesis. There would apparently be no acquired resistance to this therapy judging by the Down Syndrome data. That is important since it is widely accepted that conventional chemotherapy and hormone therapy drugs eventually cease to be effective due to acquired drug resistance.

Levels of Endostatin in normals and Down Syndrome subjects have been reported. Levels for normal controls was 20.3+/−11.5 ng/ml with range of 4 to 40. For Down Syndrome subjects, the levels were 38.6+/−20.1 ng/ml with range of 6 to 76. The sensitivity of the test kit was 2 ng/ml with typical intra- and inter-assay variances of 10% or less.

While a possible solution to the cancer problem, giving Endostatin or Endostar to every person is not likely to happen soon due to the expense and difficulty in manufacturing the drug.

The angiogenesis inhibitor Avastin has been available for a few years and has made a major impact especially in late stage colon cancer. No long term cures have been claimed from use of Avastin although the duration of survival with metastatic colon cancer is improved. Avastin inhibits VEGF, which is considered a very important angiogenic pathway in cancer. However there are many angiogenesis pathways so shutting off one pathway may not prevent angiogenesis from progressing via another pathway. Also Avastin displays some dose limiting toxicity mainly hypertension.

Based on data reported in 2003, there may well be a way to solve the breast cancer treatment problem. Mastectomies for a number of breast cancer patients and female-to-male sex change cases were used to measure angiogenesis inhibitors and promoters before and after surgery. Endostatin and VEFG were measured in plasma and wound fluid days 1 and 4 post surgery plus Endostatin baseline was measured prior to surgery. VEGF increased very significantly (9-fold) in wound fluid but not in plasma. Endostatin decreased significantly and temporarily by 20-30% in plasma but did not change in wound fluid. The Endostatin decrease appeared at day surgery+1 but then returned almost to presurgery levels by surgery+4.

According to these data, VEGF but not Endostatin is involved in wound healing. These data suggest that there are at least two important and distinct pathways for angiogenesis in early stage breast cancer. One pathway is for wound healing involving temporarily highly upregulated VEGF in the local wound area and another pathway is for systemic stimulation of tumor angiogenesis by temporarily down-regulating Endostatin. This interpretation apparently was not noticed previously. That can be taken as an indication that the method described in this application is not obvious.

Apparently, the temporary dip in naturally occurring angiogenesis inhibitors such as Endostatin is what produces the surgery-induced angiogenesis. Others mention Thrombospondin and Tumstatin as endogenous suppressors of angiogenesis in addition to Endostatin. This suggests that if the level of endogenous inhibitors such as Endostatin, Angiostatin, Tumstatin, Thrombospondin or any antiangiogenic acting protein from chromosome 21 such as NC1 in plasma could be kept high at least for those few critical days, it might prevent distant angiogenesis while not interfering with wound healing. Accordingly, Endostatin is a fragment of NC1. Although technically not endogenous, Endostar can be included in that list since it is structurally and functionally very close to Endostatin. It has been reported that Celecoxib and Indomethacin are also effective in preventing wound healing associated tumor growth so those drugs may be also considered in the list. Celecoxib was most beneficial when started 1 day before surgery in animal models. There have been some suggestions that Celecoxib may have some long term toxicity. The immunostimulant Taurolidine also can prevent surgery induced tumor growth so that drug may also be a candidate in the list although some suggest this may be a result of cells released following surgery.

Taking a clue from the Down Syndrome situation where 1.3 to 1.8 times the level of Endostatin reportedly would prevent most solid tumors over the life of the subject, an approximate value of Endostatin to retain is at least 1.3-1.8 times the level in normal subjects. The amount of Endostatin to be added will thus depend on the particular individual. Some may not need any additional Endostatin beyond the first critical few days post surgery. This would be a very effective long-term therapy for early stage breast cancer where most diagnoses are made. But to be most effective it must be started before primary surgery. Breast cancer is the most obvious, but this idea could be applied to other cancers as well. Lung cancer, melanoma, ovarian, cervical, prostate and osteosarcoma come to mind.

Since Endostar is approximately twice as effective as an antiangiogenic agent, perhaps less Endostar than Endostatin is needed.

In addition, based on known data, the effect of surgery-induced angiogenesis is not tied to removing any particular cancer but can be a sequela of general surgery. The strategy disclosed herein will apply to any cancer patient, especially early stage, who has any surgery.

The above-described therapy may be effective without any need for adjuvant chemotherapy, radiation, Herceptin for HER-2 positive patients, or adjuvant hormone therapy. The money saved by avoiding tests and not needing those modalities would help offset the costs of using Endostatin or Endostar. While it is likely unreasonable to give Endostatin or Endostar to every healthy person as a preventative, it is far more reasonable and economical to give it to every cancer patient especially if this therapy prevents relapse since that is where most of cancer care expenses occur.

Recent evidence suggests that other factors may influence whether a cancer patient relapses within five years of surgery. Accordingly, in one embodiment, a cancer patient undergoing the presently described treatment methods avoids the use of opioids, especially morphine, during the perioperative period and preferably for at least 5 years post-surgery. In a related embodiment, the cancer patient is administered the NSAID ketorolac during the perioperative period in an anesthetic capacity. Preferably, administration of ketorolac is superposed on administration of an antiangiogenic as described herein. The patient undergoing any of the treatment methods described herein may also undergo hormone aduvant or neoadjuvant treatment.

In related embodiments, a cancer patient undergoing the presently described treatment methods does not undergo transfusion or hypothermia during the perioperative period.

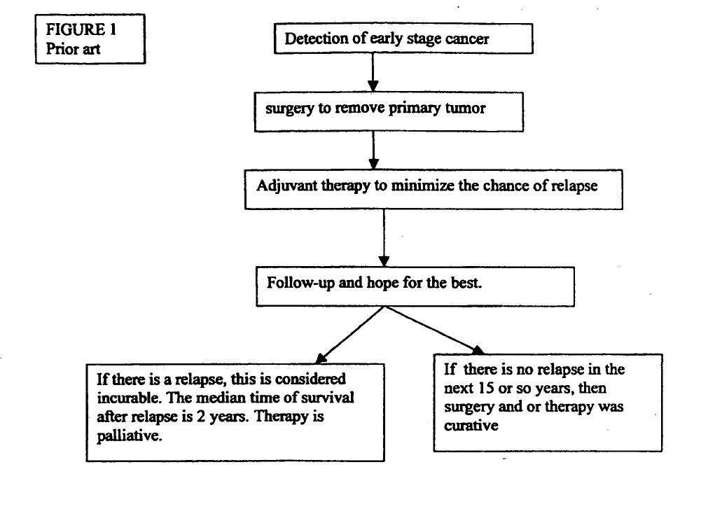

Referring now to the drawings, FIG. 1 shows prior art showing a schematic of what happens to a breast cancer patient who is newly diagnosed 1 with cancer at an early stage. That stage means there is no overt evidence of metastatic disease after full work-up including imaging and biopsy if indicated. Detection of cancer is usually by mammography but could be by other means such as a lump felt in the breast that was not there in prior times. The next step for the patient is to be sent to a surgeon who will remove 2 the primary tumor with major intent to leave no cancer behind in the breast. The surgeon will also remove one or more sentinel axillary lymph nodes for sampling on that side. If the sentinel node or nodes are clear of cancer as determined by pathology, the remainder are usually left alone. Otherwise, they are removed as well. The next step is to have the patient seen by an oncologist who will evaluate the surgical pathology report, examine the patient, have imaging studies done, run blood and genetic tests and then prescribe a treatment protocol 3. This treatment can include chemotherapy, Herceptin (if HER-2 is over expressed), hormone therapy (if estrogen or progesterone is over expressed), and radiation (especially if surgery was conservative). The patient undergoes this therapy and then the patient is followed up 4 for a number of years by the oncologist or another physician. If metastatic disease is found 5 at any site such as lung, skeleton, liver, skin, or brain, which are the usual sites, the patient is called late stage. This stage is almost always fatal. If the patient does not relapse 6 within 15 or so years after the original diagnosis, the patient is probably cured.

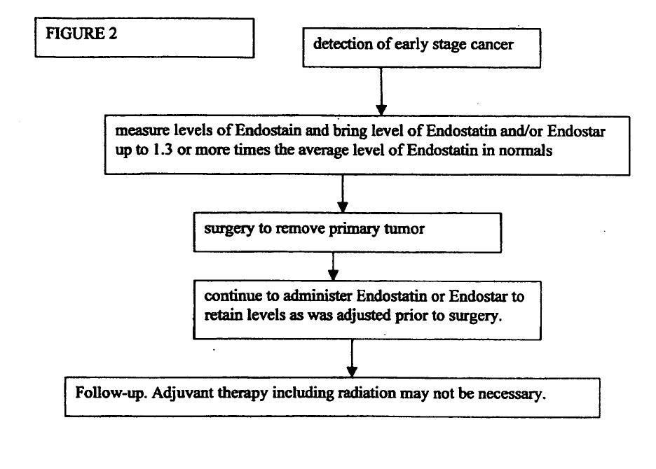

FIG. 2 shows the invention. Detection 1 of early stage breast cancer is the same as in FIG. 1 but before surgery, the patient gets tested to determine what is the level of endogenous Endostatin in her plasma. The level of Endostatin or another similar acting non-toxic angiogenesis inhibitor is then brought up 7 to a predetermined equivalent level of approximately 1.8 times the average level of Endostatin in normal persons. This is done by adding Endostar, Endostatin or any other endogenous antiangiogenic drug that is produced by genes on chromosome 21 in suitable quantity and with suitable means to keep the level at or above the desired value. Then primary surgery 2 takes place as in the prior art. After surgery, the patient continues 8 to be given Endostatin, Endostar or equivalent to retain the same levels as was indicated above for before surgery. There is no need for further therapy. Follow up 9 is still done to make sure there are no relapses.

The above-described therapy may or may not eradicate the micrometastases even if given over a long time but it could prevent growth beyond a millimeter or so indefinitely. The advantages are important. First, adjuvant chemotherapy and adjuvant hormone therapy might prove to be unnecessary since they seemingly serve to counteract surgery-induced cell division and angiogenesis. Second, with relatively long 2 year half life, avascular dormancy is a naturally very stable situation and would be far easier to maintain long term with a low toxicity antiangiogenesis inhibitor in comparison to eradicating metastases with chemotherapy, radiation, surgery or antiangiogenic therapy after they start to grow. Third, wound healing would be unimpaired while an anti-VEGF drug such as Avastin would very probably interfere with wound healing. Fourth, this therapy could be continued for ensuing years at appropriate elevated levels and may prevent future relapses for all early stage breast cancer patients. Fifth, this therapy takes full advantage of early detection and there will be no paradoxical disadvantage to anyone diagnosed early. Sixth and most important, mortality from breast cancer will be reduced. Seventh, this would be an ideal therapy for developing countries where there is a minimum of health care funds and supportive infrastructure such as medical specialists, imaging equipment and well-equipped pathology labs.

### REFERENCES CITED

### US Patent Documents

U.S. Pat. No. 5,854,205 Dec. 29, 1998 M. O'Reilly and J. Folkman

### OTHER REFERENCES

Demicheli R, Retsky M, Swartzendruber D and Bonadonna G. Proposal for a new model of breast cancer metastatic development. Annals of Oncology, 8:1075-1080, 1997.
Retsky M W, Demicheli R, Swartzendruber D E, Bame P D, Wardwell R H, Bonadonna G, Speer J, and Valagussa P. Computer simulation of a breast cancer metastasis model. Breast Cancer Research and Treatment, 45:193-202, 1997.
M Retsky, R Demicheli and W Hrushesky. Premenopausal status accelerates relapse in node positive breast cancer: hypothesis links angiogenesis, screening controversy. Breast Cancer Research and Treatment, 65:217-24, February 2001.
M Retsky, R Demicheli and W Hrushesky. Breast cancer screening for women aged 40-49 years: Screening may not be the benign process usually thought, Correspondence JNCI 93:1572, Oct. 17, 2001.
M Retsky, R Demicheli and W Hrushesky. Breast cancer screening: controversies and future directions. Current Opinion in Obstetrics and Gynecology. 15:1-8, 2003.
R Demicheli, G Bonadonna, W J M Hrushesky, M W Retsky, P Valagussa. Menopausal status dependence of the early mortality reduction due to diagnosing smaller breast cancers (T1 versus T2-T3): relevance to screening. J Clin Oncol. 22(1): 102-7, Jan. 1, 2004.
M Retsky, G Bonadonna, R Demicheli, J Folkman, W Hrushesky, P Valagussa. Hypothesis: Induced angiogenesis after surgery in premenopausal node-positive breast cancer patients is a major underlying reason why adjuvant chemotherapy works particularly well for those patients. Breast-Cancer-Research, 2004, 6:R372-R374 (14 May 2004).
R Demicheli, G Bonadonna, W J M Hrushesky, M W Retsky. P Valagussa. Menopausal status dependence of the timing of breast cancer recurrence following primary tumour surgical removal. Breast Cancer Research 2004, 6:R689-R696.
Demicheli R, Retsky M W, Hrushesky W J, Baum M, Tumor dormancy and surgery interruption of dormancy in breast cancer: learning from failures, Nature—Clinical Practice of Oncology, 4:699-710 (2007).
M. Baum, J. Cuzick, A. Howell, A. Buzdar on behalf of the ATAC Trialists' Group and R. Demicheli, M. Greco, M. Retsky, B. Hrushesky on behalf of the Milan Institute; An exploration of relapse data by hazard rate as a means of developing biological insights into the natural history and treatment of breast cancer: Data from the ‘Arimidex’, Tamoxifen, Alone or in Combination (ATAC) trial and the Milan Institute database. ASCO poster—May 2005.
Greene A K, Kim S, Rogers G F, Fishman S J, Olsen B R, Mulliken J B. Risk of vascular anomalies with Down syndrome. Pediatrics. 2008 January; 121(1):e135-40.
Wu F P, Hoekman K, Meijer S, Cuesta M A. VEGF and endostatin levels in wound fluid and plasma after breast surgery. Angiogenesis. 2003; 6(4):255-7.
Xu F, Ma Q, Sha H., Optimizing drug delivery for enhancing therapeutic efficacy of recombinant human endostatin in cancer treatment. Crit Rev Ther Drug Carrier Syst. 2007; 24(5):445-92.
Bérnard J, Béron-Gaillard N, Satgé D. Down's syndrome protects against breast cancer: is a constitutional cell microenvironment the key? Int J Cancer. 2005 Jan. 1; 113(1):168-70.
Errera F I, Canani L H, Yeh E, Kague E, Armelin-Corrêa L M, Suzuki O T, Tschiedel B, Silva M E, Sertié A L, Passos-Bueno M R. COL18A1 is highly expressed during human adipocyte differentiation and the SNP c.1136C>T in its “frizzled” motif is associated with obesity in diabetes type 2 patients. An Acad Bras Cienc. 2008 March; 80(1):167-77.
Roh J L, Sung M W, Kim K H. Suppression of accelerated tumor growth in surgical wounds by celecoxib and indomethacin. Head Neck. 2005 April; 27(4):326-32.
Hormbrey E, Han C, Roberts A, McGrouther D A, Harris A L. The relationship of human wound vascular endothelial growth factor (VEGF) after breast cancer surgery to circulating VEGF and angiogenesis. Clin Cancer Res. 2003 Oct. 1; 9(12):4332-9.
Da Costa M L, Redmond H P, Bouchier-Hayes D J. Taurolidine improves survival by abrogating the accelerated development and proliferation of solid tumors and development of organ metastases from circulating tumor cells released following surgery. J Surg Res. 2001 December; 101(2):111-9.
Kamoshita E, Ikeda Y, Fujita M, Amano H, Oikawa A, Suzuki T, Ogawa Y, Yamashina S, Azuma S, Narumiya S, Unno N, Majima M. Recruitment of a prostaglandin E receptor subtype, EP3-expressing bone marrow cells is crucial in wound-induced angiogenesis. Am J Pathol. 2006 October; 169(4):1458-72.
Lange R, Guenther T, Busch R, Hess J, Schreiber C. The presence of Down syndrome is not a risk factor in complete atrioventricular septal defect repair. J Thorac Cardiovasc Surg. 2007 August; 134(2):304-10.
Zorick T S, Mustacchi Z, Bando S Y, Zatz M, Moreira-Filho C A, Olsen B, Passos-Bueno M R. High serum endostatin levels in Down syndrome: implications for improved treatment and prevention of solid tumours. Eur J Hum Genet. 2001 November; 9(11):811-4.
Sund M, Hamano Y, Sugimoto H, Sudhakar A, Soubasakos M, Yerramalla U, Benjamin L E, Lawler J, Kieran M, Shah A, Kalluri R. Function of endogenous inhibitors of angiogenesis as endothelium-specific tumor suppressors. Proc Natl Acad Sci USA. 2005 Feb. 22; 102(8):2934-9. Epub 2005 Feb. 14.
Whitworth A. Endostatin: are we waiting for Godot? J Natl Cancer Inst. 2006 Jun. 7; 98(11):731-3.
Gottschalk A, Sharma S, Ford J, Durieux M E, Tiouririne M. The role of the perioperative period in recurrence after cancer surgery. Anesth Analg. 2010 June 110(6):1636-43.
Bovill J G, Surgery for cancer: Does anesthesia matter? Anesth Analg. 2010 June 110(6):1524-6
Holmes M D, Chen W Y, Li L, Hertzmark E, Spiegelman D, Hankinson S E. Aspirin intake and survival after breast cancer. J Clin Oncol. 2010 March 28(9):1467-72.
Forget P, Vandenhende J, Berliere M, Machiels J P, Nussbaum B, Legrand C, De Kock M. Do intraoperative analgesics influence breast cancer recurrence after masectomy? A retrospective analysis. Anesth Analg. 2010 June 110(6): 1630-5)
Nesky D M, Ambesi A, Pumiglia K M, McKeown-Longo P J. Endostatin and anastellin inhibit distinct aspects of the angiogenic process. J Exp Clin Cancer Res. 2008 November 27:61.
Keibel A, Singh V, Sharma M C. Inflammation, microenvironment, and the immune system in cancer progression. Curr Pharm Des. 2009; 15(17):1949-55.

What is claimed is:

1. A method for preventing relapse in a cancer patient undergoing surgery comprising administering a first drug that has antiangiogenic properties starting before the initial surgical incision and continuing at least until the end of surgery; and administering a second drug that has antiangiogenic properties starting within two months past the end of surgery and continuing for at least one year.

2. The method of claim 1 wherein said first drug that has antiangiogenic properties is a non-steroidal anti-inflammatory drug (NSAID).

3. The method of claim 2, wherein the NSAID is selected from the group consisting of Ibuprofen, Naproxen, Fenoprofen, Ketoprofen, Flurbiprofen, Oxaprozin, Indomethacin, Sulindac, Etodolac, Ketorolac, Diclofenac, Nabumetone, Piroxicam, Meloxicam, Tenoxicam, Droxicam, Lornoxicam, Isoxicam, Mefenamic acid, Meclofenamic acid, Flufenamic acid, Tolfenamic acid, Aspirin, and Acetaminophen.

4. The method of claim 3 wherein the NSAID is Ketorolac.

5. The method of claim 1, wherein the patient is early stage.

6. The method of claim 1, wherein the patient has breast cancer.

7. The method of claim 1, wherein the patient has cancer other than breast cancer.

8. The method of claim 1, wherein the surgery is to remove a primary tumor.

9. The method of claim 1, wherein the surgery is for any reason other than to remove a primary tumor.

10. The method of claim 1, wherein said relapse is metastatic.

11. The method of claim 1, wherein said relapse is local.

12. The method of claim 1 wherein said second drug that has antiangiogenic properties is a non-steroidal anti-inflammatory drug (NSAID).

13. The method of claim 12, wherein the NSAID is selected from the group consisting of Ibuprofen, Naproxen, Fenoprofen, Ketoprofen, Flurbiprofen, Oxaprozin, Indomethacin, Sulindac, Etodolac, Ketorolac, Diclofenac, Nabumetone, Piroxicam, Meloxicam, Tenoxicam, Droxicam, Lornoxicam, Isoxicam, Mefenamic acid, Meclofenamic acid, Flufenamic acid, Tolfenamic acid, Aspirin, and Acetaminophen.

14. The method of claim 1 wherein the second drug that has antiangiogenic properties is a selective estrogen receptor modulator (SERM) and/or aromatase inhibitor.

15. The method of claim 14, wherein said SERM is selected from the group consisting of clomiphene, femarelle, ormeloxifene, raloxifene, tamoxifen, toremifene, afimoxifene, arzoxifene, bazedoxifene, and lasofoxifene.

16. The method of claim 14, wherein said aromatase inhibitor is selected from anastrozole, exemestane, and letrozole

17. The method of claim 1, wherein said first drug that has antiangiogenic properties is selected from endostatin, angiostatin, Endostar, tumstatin and thrombospondin.

18. The method of claim 17, wherein said first drug is Endostatin.

19. The method of claim 18, wherein the Endostatin is taken to a level in plasma greater than 24.36 ng/ml before the start of surgery.

20. The method of claim 7, wherein the first drug that has antiangiogenic properties is Endostar.

21. The method of claim 20, wherein the Endostar is taken to a level in plasma greater than 24.36 ng/ml.

22. The method of claim 1, wherein the first drug that has antiangiogenic properties is the same as the second drug that has antiangiogenic properties.

23. The method of claim 1, wherein hypothermia is avoided in the patient during the perioperative period.

24. The method of claim 1 wherein said second drug with antiangiogenic properties is administered in a metronomic schedule.

25. The method of claim 1 wherein the use of morphine is avoided.

26. The method of claim 1 wherein the first said drug that has antiangiogenic properties is an analgesic.
